(19) United States
(12) Patent Application Publication
(10) Pub. No.: US 2017/0087299 A1
(43) Pub. Date: Mar. 30, 2017

(54) CARRIER APPARATUS TO DELIVER A SUBSTANCE AND METHODS THEREOF

(71) Applicant: Glen J. Anderson, Beaverton, OR (US)

(72) Inventor: Glen J. Anderson, Beaverton, OR (US)

(73) Assignee: Intel Corporation, Santa Clara, CA (US)

(21) Appl. No.: 14/866,668

(22) Filed: Sep. 25, 2015

(51) Int. Cl. A61M 5/172; A61B 5/00; A61B 5/145

(52) U.S. Cl. CPC A61M 5/1723; A61B 5/14503; A61B 5/4848; A61M 2005/1726; A61M 2205/50

(57) ABSTRACT

Systems, apparatuses, kits, and methods may provide a substance. A carrier apparatus may include a reservoir to hold a payload, a sensor to detect a target in a subject and generate data corresponding to the target, a processor to allow the payload to be accessible from the reservoir based on the data, and/or a biocompatible carrier composition to hold the reservoir, the sensor, and/or the processor. The reservoir may include a placebo reservoir, a medication reservoir, a diagnostic reservoir, and/or a modification reservoir. The payload may be made available based on a diagnosis, a detection of a target in a body, a level of the target in the body, and/or a context in which the carrier apparatus is deployed. The carrier apparatus may also include a wireless rechargeable battery to power a component of the carrier apparatus. The carrier apparatus may provide diagnostic and/or therapeutic functionality.

### TECHNICAL FIELD

Embodiments generally relate to a carrier apparatus. More particularly, embodiments relate a carrier apparatus including diagnostic and therapeutic functionality.

### BACKGROUND

Drug systems may include pills with a computer chip transmitting a time that a drug was ingested to overcome forgetful impulses. Pills may also include micro-needles to deliver a drug with the goal of replacing injections. In addition, nanoparticles may deliver a drug into cells when exposed to oscillating magnetic fields. Implantable devices may also deliver a drug, such as insulin. Such systems, however, may require interaction with devices external to a subject and/or user input to operate. Also, such systems may provide incorrect or incomplete therapeutic responses. For example, dose response may be based on a passive state such as physiological pH, user-based actions, or actions that do not account for a plurality of conditions and/or symptoms.

Meanwhile, detection systems may include imaging equipment (e.g., CT scan, endoscopy, colonoscopy, camera pill, etc.) to image a sample, bioassays using living cells, capture antibodies, etc., to detect an analyte in a sample, Lab-on-chip technologies to detect an analyte in a sample, and so on. Such systems, however, may require interaction with devices external to a subject and/or user input to operate. For example, specialized equipment or expertise may be needed to handle samples, execute a reaction, and/or evaluate a diagnosis. Also, such systems may provide stale information or incomplete information that may lead to incorrect or incomplete therapeutic responses.

### BRIEF DESCRIPTION OF THE DRAWINGS

The various advantages of the embodiments will become apparent to one skilled in the art by reading the following specification and appended claims, and by referencing the following drawings, in which:

FIG. 1 is an illustration of an example of a carrier apparatus to deliver a substance according to an embodiment;

FIG. 2 is a flowchart of an example of a method to deliver a substance according to an embodiment;

FIG. 3 is a block diagram of an example of a processor according to an embodiment; and

FIG. 4 is a block diagram of an example of a computing system according to an embodiment.

### DESCRIPTION OF EMBODIMENTS

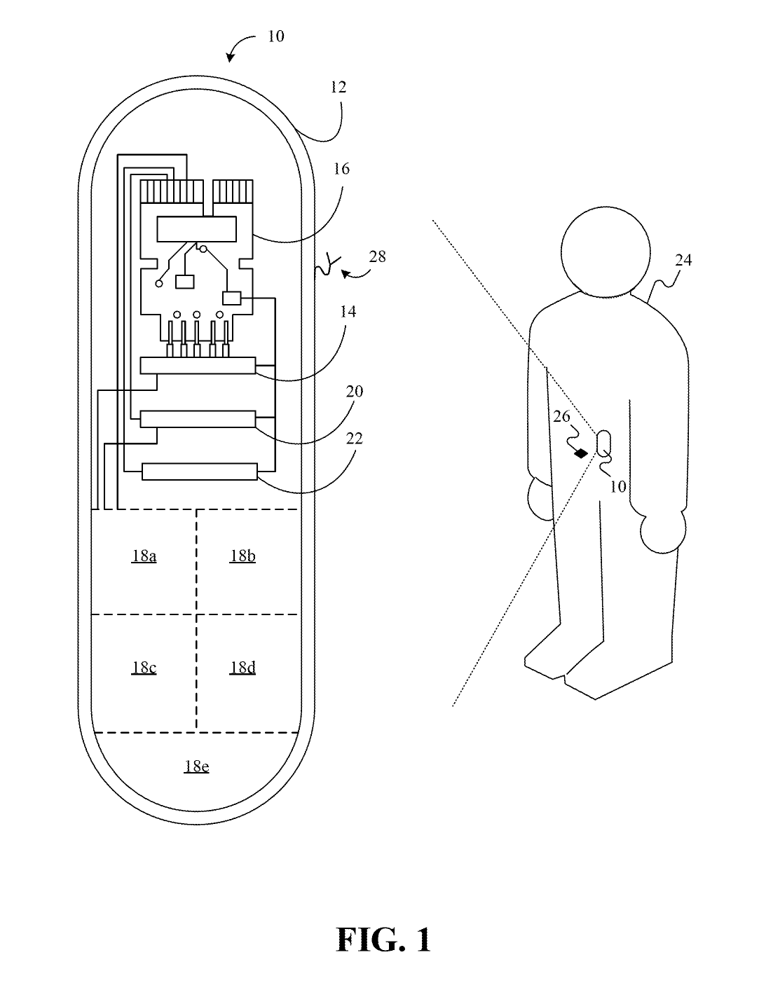

Turning now to FIG. 1, a carrier apparatus 10 is shown to deliver a substance that includes a biocompatible carrier composition 12 to hold one or more components such as a sensor 14, a processor 16, a reservoir 18 (18a-18e), a pump 20, and/or a power source 22. The carrier apparatus 10 may provide diagnostic and/or therapeutic functionality. In particular, the carrier apparatus 10 may be administered to a subject 24 to diagnose a condition by detecting a target 26, which may include a causative analyte creating the condition (e.g., bacteria, cancer, acid, etc.), a marker analyte that indicates the presence or likelihood of the condition (e.g., a bio-marker, etc.), a reporter analyte that indicates the presence of the causative analyte or the marker analyte (e.g., fluorescent reporter, etc.), and so forth. In addition, the carrier apparatus 10 may be administered to the subject 24 to prevent and/or treat the condition based on, for example, the diagnosis. The subject may include, for example, a human, and animal, and so forth.

The carrier composition 12 may include an implantable composition, an ingestible composition, an injectable composition, a topical composition, and so forth. For example, the carrier composition 12 may include a pharmaceutically acceptable carrier such as a non-toxic carrier, a physiologically acceptable carrier, and so forth. In one example, the carrier composition 12 may only hold payload (e.g., diagnostic payload, etc.) and include an emulsion, a paste, cream, a lotion, a gel, a jelly, an ointment, an oil, an aerosol, a powder, a solvent, a liposome, a micelle, a peptide (e.g., albumin), etc.

In another example, the carrier composition 12 may hold a component of the carrier apparatus 10 and include a synthetic polymer (e.g., polyglycolides, polylactides, and their copolymers), a natural polymer (e.g., hyaluronic acid, dextran, chitosan), a 2-dimensional nanomaterial (e.g., a film), a 3-dimensional material (e.g., polymeric matrix such as polyethylene glycol (PEG)), a tissue compatible material (e.g., a metal, a polymer such as poly(propylene fumarate), polyanhydrides, polycarbonates, polyurethanes, polyphosphazenes, etc.), a capsule (e.g., a hard-shelled capsule, soft-shelled capsule, etc.), a tablet, and so forth. In the illustrated example, the carrier composition 12 includes a capsule that is ingested by the subject 24 to detect a condition (e.g., a malady), to gather and/or provide information (e.g., over a period of time), and so forth. In this regard, the carrier composition 12 may hold a network communication device (not shown) to provide data to an external (to the body) computing system that may track the carrier apparatus 10, store information over a period of time to allow for analysis of the data for diagnostic and/or therapeutic purposes, provide activation, and so forth.

Thus, the carrier apparatus 10 may include communication functionality for a wide variety of purposes such as, for example, cellular telephone (e.g., Wideband Code Division Multiple Access/W-CDMA (Universal Mobile Telecommunications System/UMTS), CDMA2000 (IS-856/IS-2000), etc.), WiFi (Wireless Fidelity, e.g., Institute of Electrical and Electronics Engineers/IEEE 802.11-2007, Wireless Local Area Network/LAN Medium Access Control (MAC) and Physical Layer (PHY) Specifications), 4G LTE (Fourth Generation Long Term Evolution), Bluetooth (e.g., Institute of Electrical and Electronics Engineers/IEEE 802.15.1-2005, Wireless Personal Area Networks), WiMax (e.g., IEEE 802.16-2004, LAN/MAN Broadband Wireless LANS), Global Positioning System (GPS), spread spectrum (e.g., 900 MHz), NFC (Near Field Communication, ECMA-340, ISO/IEC 18092), and other radio frequency (RF) purposes.

In addition, the carrier composition 12 may be linked with (e.g., covalently linked, electrostatically linked, etc.) and/or may physically sequester (e.g., physically trap) one or more substances, such as payload, the target 26, a ligand 28 specific for the target 26, and so forth. In the illustrated example, the carrier composition 12 is linked or otherwise associated with the ligand 28 functionalizing an external portion of the carrier apparatus 10, such as an external surface interfacing with the gastrointestinal (GI) system of the subject 24. The ligand 28 (or another ligand) may also functionalize an internal portion of the carrier apparatus 12, such as an inner sidewall of the carrier apparatus 10, an inner microfluidic channel of the carrier apparatus 10, one or more components of the carrier apparatus 10 such as the sensor 14, and so forth. Thus, sample having the target 26 such as digestive fluid, intestinal fluid, interstitial fluid, blood, etc., may diffuse through the carrier composition 12 or be pumped into and/or through the carrier apparatus 10 by the pump 20 for coupling with the ligand 28 and detection by the sensor 14.

The sensor 14 may include any sensor to detect an analyte. In one example, the sensor 14 may include an electrical sensor to generate and/or measure a current induced or modified when the target 26 is coupled with the ligand 28. For example, a charge from the bound target 26 may cause a channel to appear (or disappear) between a source and a drain (e.g., conductor material, etc.) of a transistor (e.g., doped semiconductor material, etc.). The sensor 14 may also generate and/or measure a voltage that is induced or modified when the ligand 28 couples with the target 26. In addition, the electrical signals may be generated in a concentration-dependent manner. For example, a current level or a voltage level may be proportional to an amount of analyte.

In another example, the sensor 14 may include a piezoelectric sensor (e.g., quartz material, etc.) to measure changes in pressure caused by the target 26 and convert the changes to an electrical signal (e.g., a voltage, etc.) in proportion to compressive or tensile mechanical stress or strain. For example, the sensor 14 may measure a change in pressure caused by an analyte (e.g., a gas) to detect a condition (e.g., indigestion, bloating, risk of ulcer, etc.). In a further example, the sensor 14 may include an optical sensor to measure changes in light intensity caused by specific groups of the target 26. For example, a light emitting diode-based microfluidic detector (e.g., metal-oxide-semiconductor fluorescence detector) may generate an optical signal from changes in light intensity caused by a fluorescence reporter (e.g., glucose-responsive fluorescent hydrogel, detection antibody, quantum dot, bead, etc.). The sensor 14 may be omitted or inactive when, for example, the carrier apparatus 10 makes payload available in response to a passive state such as physiological pH, temperature, passage of time, etc.

The processor 16 may allow payload to be accessible from the reservoir 18 based on data (e.g., an electrical signal, an optical signal, etc.) generated by the sensor 14 that corresponds to the target 26. For example, the processor 16 may receive the data and cause the pump 20 to draw payload from the reservoir 18 (e.g., through a microfluidic channel) to allow for payload release from the carrier apparatus 10. The processor 16 may also actuate a valve in the carrier apparatus 10 between the reservoir 18 and the subject 24 to allow for payload release from carrier apparatus 10 whether or not the pump 20 is implemented. The processor 16 may further utilize the pump 20 and/or the valve to allow sample to enter a reactor area and/or the reservoir 18 of the carrier apparatus 10. Also, the processor 16 may prevent an action from occurring that makes payload accessible, such as preventing timed release of payload, preventing activation of payload, causing the power source 22 to restrict power to one or more components, and so forth.

In addition, the processor 16 may evaluate an amount of target 16 (e.g., concentration of an analyte) and allow an amount of payload to be released from the carrier apparatus 10 conditional on the amount of the target 16 (e.g., conditional release). For example, the processor 16 may utilize microcode to apply a dosage scale for detected amounts of the target 16 to provide precise conditional release of specific payload based on real-time and/or dynamic diagnosis. The processor 16 may also allow the release of payload when the data indicates the presence of the target 26. The processor 16 may be omitted or inactive when, for example, the carrier apparatus 10 makes payload available in response to a passive state such as physiological pH, temperature, passage of time, etc.

Payload made accessible by the carrier apparatus 10 may be located in the reservoir 18 (18a-18e), which holds any substance involved in addressing (e.g., diagnosing, preventing, treating, etc.) a condition of the subject 26. Thus, the carrier apparatus 10 may be provided to address any condition or combination of conditions (including symptoms thereof) with one or more payloads. For example, the reservoirs 18a, 18b may hold a medication payload to address a condition associated with the target 26, the reservoir 18c may hold a diagnostic payload to augment detection of the target 26 via the diagnostic payload to address a condition associated with the target 26, the reservoir 18d may hold a modification payload to dissolve at least a portion of the carrier apparatus 10 to address a condition associated with the target 26, the reservoir 18e may hold a placebo to test effectiveness (e.g., efficacy, toxicity, etc.) of a substance to address a condition associated with the target 26, etc.

In one example, the reservoir 18a (e.g., first medication reservoir) may hold a first medication payload to be released from the reservoir 18a to address a first condition associated with the target 26. For example, the reservoir 18a may hold an anti-inflammatory drug (e.g., a nonsteroidal anti-inflammatory drug (NSAID), a steroid, etc.) to address inflammation when C-reactive protein (CRP) is detected by the sensor 14. In another example, the reservoir 18b (e.g., second medication reservoir) may hold a second medication payload to be released from the reservoir 18b to address the first condition associated with the target 26. For example, the reservoir 18b may hold an anti-inflammatory drug (e.g., a NSAID, a steroid, etc.) to also address inflammation when CRP is detected by the sensor 14.

Additionally, the reservoir 18b may hold a second medication payload to be released from the reservoir 18b to address a second condition associated with the target. For example, the reservoir 18b may hold a heart disease drug (e.g., angiotensin converting enzyme (ACE) inhibitors, beta-blockers, calcium channel blockers, etc.) to address heart disease when CRP is detected by the sensor 14. The reservoir 18b may also hold a second medication payload to be released from the reservoir 18b to address a third condition associated with another target. For example, the reservoir 18b may include an inflammatory bowl disease (IBD) drug (e.g., an aminosalicylate, a corticosteroid, an immune system suppressor, an antibiotic, an anti-diarrheal, a pain reliever, an iron supplement, vitamin B-12, etc.) to address a specific form of IBD such as ulcerative colitis and/or Crohn's disease when an antibody is detected by the sensor 14 including, for example, perinuclear anti-neutrophil cytoplasmic antibody, Saccharomyces Cerevisiae antibodies, Clostridium species antibodies, Escherichia coli antibodies, Pseudomonas fluorescens antibodies, calprotectin, lactoferrin, and so forth.

In another example, the reservoirs 18a, 18b may hold a medication payload to be released from the reservoirs 18a, 18b to address a cancer condition associated with the target 26. For example, the reservoir 18a may hold an anti-cancer drug (e.g., chemotherapeutic, etc.) to address cancer when a bio-marker for cancer is detected. The sensor 14 may detect, for example, a signature protein such as vascular endothelial growth factor (VEGF) that is released by many solid tumors including gastric carcinoma. In response to the diagnosis, the carrier apparatus 10 may release an anti-cancer drug to target one or more actors in the cancer pathway via, for example, the processor 16. Thus, the reservoirs 18a, 18b may hold kinase inhibitors, agonists, monoclonal antibodies, antagonists, etc., to target c-MET, the reservoirs 18a, 18b may hold taxol, nanoparticles with anti-cancer drugs, etc., to target cell proliferation, and so forth.

In a further example, the sensor 14 may directly detect H. pylori or may detect a product of H. pylori such as vaculating cytotoxin A (Vac-A). For example, H. pylori may couple with the ligand 28 for detection by the sensor 14. The reservoirs 18a, 18b may therefore hold a medication payload to be released to treat gastritis, duodenal ulcers, and/or gastric carcinomas in response to the detection of H. pylori, products thereof, or markers thereof. In addition, the reservoirs 18a, 18b may hold a medication payload to be released to minimize H. pylori such as antibiotics, antibodies, and so forth. H. pylori may be treated with, for example, proton pump inhibitors (e.g., omeprazole) and antibiotics (e.g., clarithromycin, amoxicillin, etc.). Another therapeutic agent may also be administered from the reservoirs 18a, 18b automatically on encountering H. pylori (and/or products or markers thereof) such as an antacid/anti-diarrhea (e.g., bismuth, etc.), an anticoagulant (e.g., for an ulcer or likelihood of an ulcer detected via, e.g., pH), H2 blockers (e.g., rantidine, famotidine, etc.), and so forth.

The reservoir 18c (e.g., diagnostic reservoir) may hold a diagnostic payload to augment detection of the target 26. In one example, the reservoir 18c may include a reporter diagnostic reservoir to hold a reporter payload to be coupled with the target 26 for detection by the sensor 14 via the reporter. For example, the reservoir 18c may hold a fluorescence reporter to couple with the target 26 for detection by the sensor 14. In this regard, the fluorescence reporter may be released from the reservoir 18c into the subject 24 or may be released into another area of the carrier apparatus 10 such as a reactor area (e.g., microfluidic channel, mixing reservoir, etc.) to facilitate a coupling reaction between the target 26 and the fluorescence reporter.

The pump 20 may, for example, introduce sample to the reservoir 18c to facilitate a coupling reaction and allow the reservoir 18c to release the target 26 coupled with the fluorescence reporter for detection by the sensor 14. Thus, the target 26 coupled with the fluorescence reporter or the target 26 (e.g., to be coupled with the fluorescence reporter) may, for example, diffuse through the carrier composition 12 or be pumped into and/or through the carrier apparatus 10 for detection by the sensor 14. The target 26 coupled with the fluorescence reporter or the target 26 (e.g., to be coupled with the fluorescence reporter) may also couple with the ligand 28 for detection by the sensor 14.

In another example, the reservoir 18c may include a synthesis diagnostic reservoir to hold a synthesis payload to couple with the target 26 to synthesize the reporter. For example, the reservoir 18c may hold a chemical reporter (e.g., urea) to couple with H. pylori and catalyze the production of carbon dioxide (CO₂). The urea may include a non-toxic label (e.g., an isotope of carbon, etc.) that may be detected from the CO₂ produced by H. pylori. The CO₂ may then, for example, diffuse through the carrier composition 12 or be pumped into and/or through the carrier apparatus 10 for detection by the sensor 14. H. pylori may also diffuse through the carrier composition 12 or be pumped into and/or through the carrier apparatus 10 for detection by the sensor 14. In this regard, urea may be fed into a mixing chamber or H. pylori may be fed into the reservoir 18c holding urea to catalyze the synthesis of the reporter (e.g., CO₂).

In a further example, the reservoir 18c may hold a bacterial reporter including live cells that couple with specificity to materials (e.g., bio-markers, etc.) and synthesize one or more reporters via transcription and translation processes for detection by the sensor 14. Thus, the target 26 coupled with the bacterial reporter, the target 26 (e.g., to be coupled with the bacterial reporter), and/or the synthesized reporter may, for example, diffuse through the carrier composition 12 or be pumped into and/or through the carrier apparatus 10 for detection by the sensor 14. Moreover, the synthesized reporter may couple with the ligand 28 for detection by the sensor 14.

In yet another example, the reservoir 18c may include an amplifier diagnostic reservoir to hold an amplifier payload to couple with the target 26 and amplify a characteristic of the target 26 for detection by the sensor 14 via the characteristic. For example, the reservoir 18c may hold a ligand (e.g., antibody, synthetic small molecule, etc.) that binds to the target 26 with specificity and/or affinity to enhance an electrical signal caused by the target 26. In this regard, the properties of the target 26 may be considered (e.g., polar charge, hydrophobicity, pKa, etc.) to select and/or customize the properties of the amplifier payload for amplification of the signal caused by the target 26.

For example, a negatively charged target may be coupled with an amplifier payload that amplifies the change in a current or a voltage caused by the negatively charged target by selecting or customizing the amplifier payload with one or more negatively charged residues or groups. Thus, the target 26 coupled with the amplifier payload or the target 26 (e.g., to be coupled with the amplifier payload) may, for example, diffuse through the carrier composition 12 or be pumped into and/or through the carrier apparatus 10 for detection by the sensor 14. Moreover, target 26 coupled with the amplifier payload may bind to the ligand 28 for detection by the sensor 14.

The reservoir 18d (e.g., modification reservoir) may hold a modification payload to be released from the reservoir 18d to modify at least a portion of the carrier apparatus 10 to release payload such as a medication payload, a diagnosis payload, a placebo payload, and so forth. In one example, the reservoir 18d may include a coating modification reservoir to hold a coating modification payload to modify a coating of the carrier apparatus 10. For example, the reservoir 18d may hold an acid or a base (e.g., relative to physiological pH) to be released to dissolve a pH responsive coating such as an acrylic coating. Thus, the modification payload may be implemented for timed or immediate payload release, and more granular control in response to a specific condition.

In another example, the reservoir 18d may include a matrix modification reservoir to hold a matrix modification payload to be released from the reservoir 18d to modify a matrix of the carrier apparatus 10. For example, the reservoir 18d may hold payload to modify (e.g., swell, de-swell, dissolve, etc.) a stimuli responsive hydrogel in response to changes in pH, temperature, ionic strength, electromagnetic radiation, etc. In one example, the reservoir 18d may hold a solution having ions to swell or de-swell a hydrogen membrane (e.g., natural or synthetic) based on induced ionic strength changes. Thus, the modification payload may be released from the reservoir 18d into the subject 24 or may be released into another area of the carrier apparatus 10 to facilitate modification of at least a portion of the carrier apparatus 10, such as a sidewall of the reservoir 18, a portion of the carrier composition 12, and so on.

The reservoir 18e (e.g., placebo reservoir) may hold a placebo payload to be released from the reservoir 18e as a placebo in a clinical trial. In one example, the reservoir 18e may include a substance with no therapeutic effect that is to be released to test the effectiveness of another substance (e.g., drug or combination of drugs, reporters, amplifiers, etc.) in a randomized clinical trial. Notably, the processor 16 may determine a context in which the carrier apparatus 10 is to be deployed and allow payload to be released from the reservoir 18 based on the context.

In one example, the processor 16 may determine, in real-time or based on a pre-selection, whether the carrier apparatus 10 is to release a medication or a placebo in a clinical trial to maximize randomness and minimize bias. In another example, the processor 16 may determine, in real-time or based on a pre-selection, that the carrier apparatus 10 is to provide timed or immediate release and perform suitable actions such as implementing the pump 20, actuating a valve, allowing release of a medication payload, a diagnostic payload, a modification payload, and so forth.

In a further example, the processor 16 may determine a current or predicted future state of the subject 24. Contexts may therefore include physical states such as running, walking, sleeping, or sitting. Contexts may also include locations, such as restaurants, bars, health-care provider locations, and so forth. Contexts may further include more specific situations such as attending a particular meeting, going to the dentist's office, driving, and so forth. Contexts may also include biometric states such as blood glucose levels, pain, heart function, and so forth.

The processor 16 may determine and/or anticipate situations in which payload may be needed to ensure sufficient time to create a therapeutic effect to combat undesirable effects on the subject 26 caused by a situation. For example, the processor 16 may determine that the user is going to a dentist's office and the system may activate an anti-anxiety medication prior to arriving such that the user is relaxed upon arrival. The processor 16 may utilize information about how much time it takes for payload to achieve its effects (e.g., a therapeutic effect) as well as information on a schedule of the subject 24 to time activation to achieve a full effect at the start of the dentist appointment. The processor 16 may also evaluate a risk of developing a condition or a symptom thereof and respond accordingly. The sensor 14 may be omitted, or may be utilized to detect the target 26 (e.g., endorphin, etc.), when a therapeutic response is context-based.

Moreover, the carrier apparatus 10 may make payload available in an inactive or less than fully active form to be activated when desired or needed. In one example, the medication reservoirs 18a, 18b may include medication payload (e.g., in nanoparticles) to be activated via an RF magnetic field (e.g., using a wearable device, from the carrier apparatus, etc.). Activation may deliver payload on demand in response to, for example, a context. In another example, the medication reservoirs 18a, 18b may include a prodrug (e.g., a drug precursor) that is transformed (e.g., in vivo) to provide the active form for an intended pharmacological effect, by various mechanisms (e.g., metabolic process, chemical process, etc.) such as, for example, through hydrolysis in fluid (e.g., blood, cytoplasm, in a compartment of a carrier apparatus, etc.). For example, a protective group (e.g., non-toxic protective group) may be utilized in a transient manner, which may be directly linked or indirectly coupled (e.g., via a succinic spacer, an amino acid spacer, as a liposome, micelle, etc.) with medication payload (e.g., via a functional group, sequestering the payload, etc.) before or after loading into the carrier apparatus 10 and removed by intracellular or extracellular mechanisms for activation.

Additionally, a component of the carrier apparatus 10 may be distributed in a plurality of systems. For example, the carrier composition 12 may only hold a payload such as a diagnostic payload when, for example, two or more systems are implemented. In one example, the carrier composition 12 may only hold a diagnostic payload and include an emulsion, a paste, cream, a lotion, a gel, a jelly, an ointment, an oil, an aerosol, a powder, a solvent, a liposome, a micelle, a peptide (e.g., albumin), etc. Thus, for example, the carrier composition 12 may include a capsule or a tablet that only holds a diagnostic payload to be administered ahead of the carrier apparatus 12 to facilitate a more complete diagnosis and correct therapeutic effect by allowing sufficient time for augmentation of the signal caused by one or more targets.

The power source 22 of the carrier apparatus 10 may provide power to one or more components including, for example, the sensor 14, the processor 16, the pump 20, and so forth. In one example, the power source 22 may include a battery, such as a nickel cadmium battery for relatively short-term use, a lithium ion battery for relatively long-term use, and so forth. In another example, the power source 22 may include a rechargeable battery such as a wireless rechargeable battery that may be re-charged via a charging material (e.g., magnetic material forming a charging coil, etc.) and/or a charging device such as a wearable charging cuff, a charging pad, etc.

Additionally, the carrier apparatus 10 may be maintained in a container of a kit formed of any suitable material. For example, the container may be formed of a metal material, a polymeric material (e.g., plastic), and so on. The container may include a dispenser portion, such as an opening that is accessed via a removable cap (e.g., a threaded cap), a nozzle (e.g., an inhaler nozzle), a removable film (e.g., a patch film), a perforated surface (e.g., a tablet package perforated surface), an injection surface (e.g., a fluid drawing surface of a vial), and so forth. Thus, the container may include a chamber to maintain the carrier apparatus 10, which may be accessed via the dispenser portion.

The container may be associated with an instruction regarding the carrier apparatus 10, such as a storage instruction (e.g., a storage condition), a use instruction (e.g., administration regimen, implant process, etc.), a disposal instruction, a warning, and so forth. The container may also be associated with information regarding the carrier apparatus 10, such as a chemical formula of payload, a structural formula of payload, a property (e.g., molecular weight, melting point, concentration, etc.) of payload, an expiration date of the carrier apparatus 10, a listing of components of the carrier apparatus 10, and so forth.

The container may include a label to provide the instruction and/or the information regarding the carrier apparatus 10. The instruction and/or the information may also be accessible from data storage, such as a computer server, computer readable medium, a database structure, and so on. For example, the instruction and/or the information may be in any data format, such as a text editor format (RTF), an image format (JPEG), a portable document format (PDF), a markup language format (HTTP, XML), a spreadsheet format, and so on.

Accordingly, the container may be a pill bottle, an inhaler package, a transdermal patch package, an eye drop bottle, a vial, a box, and so forth. In this regard, a label may be physically attached or otherwise associated with the container to provide instructions related to, e.g., an administration regimen, fabrication instructions, etc. The container may also be a laboratory storage container, a transport container, and so forth. In this regard, a label may be physically attached or otherwise associated with the container to provide instructions related to, e.g., suitable storage conditions (e.g., pressure, temperature), hazard warnings, and so forth. Thus, a kit may provide a diagnostic system (e.g., via a conventional capsule, via a carrier apparatus including only a diagnostic payload, etc.) and a separate therapeutic system including, e.g., the sensor 14, the processor 16, and/or payload in the reservoir 18 (e.g., 18a, 18b, etc.) with instructions and/or information via a label, data storage device (e.g., memory), etc.

The carrier apparatus 10 may therefore provide precise conditional release of specific payload based on real-time and/or dynamic diagnosis to ameliorate drawbacks caused by, e.g., NSAIDs including bleeding, antibodies including antibiotic resistance, danger to GI flora, and so forth. In addition, the carrier apparatus 10 may provide precise conditional release of specific payload based on real-time and/or dynamic diagnosis to customize treatment under the circumstances. For example, the subject 24 may orally ingest the carrier apparatus 10 to deliver a medication payload in an effective amount as it travels through the GI system immediately upon analyte detection or at predetermined times or locations to minimize degradation or maximize adsorption, distribution, etc.

The delivery of a therapeutic agent may be based on a dynamic diagnosis (including differential diagnosis of), e.g., an ulcer, inflammation, IBD type, and so forth. In this regard, a more granular level of diagnosis and/or treatment may be provided with non-invasive (e.g., smart pill) and/or minimally invasive (e.g., suppository, subcutaneous implant, etc.) systems. Thus, the carrier apparatus 10 may include diagnostic and/or therapeutic (e.g., prevention, treatment, etc.) functionality to provide a more complete (e.g., global, differential) diagnosis and/or a correct and complete therapeutic response.

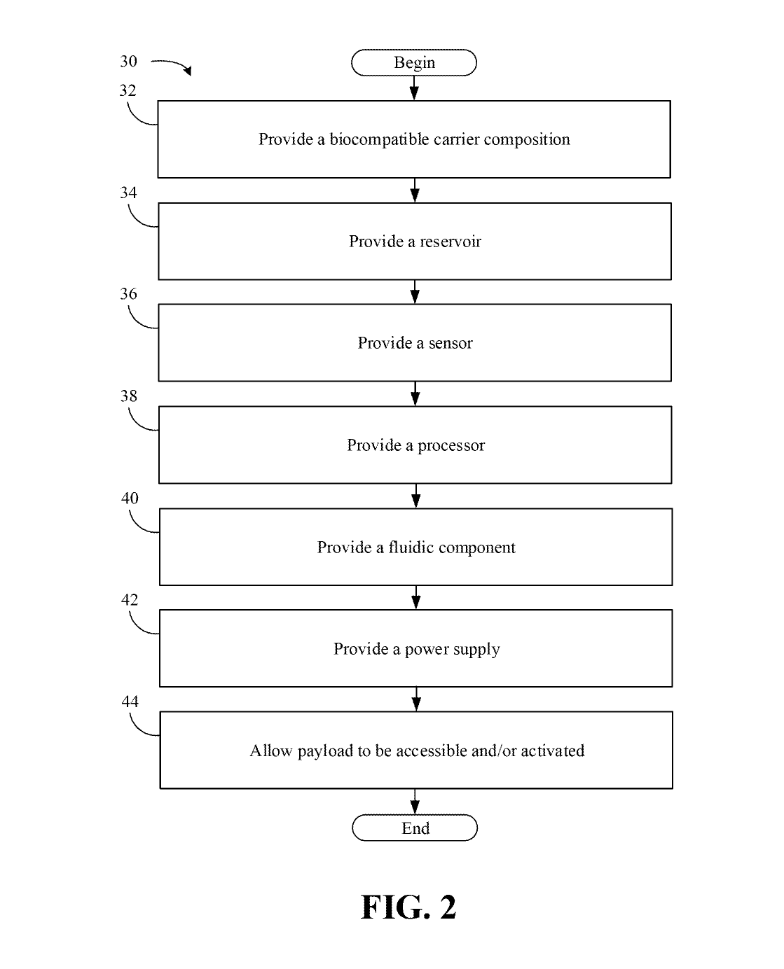

FIG. 2 shows a method 30 to deliver a substance. The method 30 may be implemented as a module or related component in a set of logic instructions stored in a non-transitory machine- or computer-readable storage medium such as random access memory (RAM), read only memory (ROM), programmable ROM (PROM), firmware, flash memory, etc., in configurable logic such as, for example, programmable logic arrays (PLAs), field programmable gate arrays (FPGAs), complex programmable logic devices (CPLDs), in fixed-functionality hardware logic using circuit technology such as, for example, application specific integrated circuit (ASIC), complementary metal oxide semiconductor (CMOS) or transistor-transistor logic (TTL) technology, or any combination thereof. For example, computer program code to carry out operations shown in the method 30 may be written in any combination of one or more programming languages, including an object oriented programming language such as JAVA, SMALLTALK, C++ or the like and conventional procedural programming languages, such as the “C” programming language or similar programming languages.

Illustrated processing block 32 provides a biocompatible carrier composition of a carrier apparatus, such as an in ingestible carrier composition of an ingestible carrier apparatus (e.g. a capsule, etc.), an implantable carrier composition of an implantable carrier apparatus (e.g., an implant, etc.), and so forth. Block 32 may involve the fabrication and/or the use of the biocompatible carrier composition. For example, the formation of the biocompatible carrier composition may include conventional chemical synthesis processes (e.g., polymer chemical synthesis, etc.), molding processes, pick-and-place processes, lithography processes (e.g., photolithography, etching, etc.), printing processes (e.g., screen printing, three-dimensional printing, etc.), lamination processes, coating processes (e.g., dip coating, etc.), deposition processes (e.g., chemical vapor deposition, physical vapor deposition, electro-deposition, etc.), and so forth. The utilization of the biocompatible carrier composition may include the administration and/or the implementation of one or more biocompatible carrier compositions via one or more carrier apparatuses to provide diagnostic and/or therapeutic functionality. In addition, block 32 may also involve the packaging of one or more carrier apparatuses including one or more biocompatible carrier compositions in a container (e.g., of a kit).

Illustrated processing block 34 provides a reservoir, for example in the biocompatible carrier composition, of the carrier apparatus. The reservoir may hold a payload to address a condition, such as a medication reservoir to hold a medication payload, a diagnostic reservoir to hold a diagnostic payload, a modification reservoir to hold a modification payload, a placebo reservoir to hold a placebo payload, and so forth. Block 34 may involve the fabrication and/or the use of the reservoir. For example, the formation of the reservoir may include conventional chemical synthesis processes, molding processes, pick-and-place processes, lithography processes, printing processes, lamination processes, coating processes, deposition processes, and so forth. The utilization of the reservoir may include the administration and/or the implementation of one or more reservoirs via one or more carrier apparatuses to provide diagnostic and/or therapeutic functionality. In addition, block 34 may involve the packaging of one or more carrier apparatuses including one or more reservoirs in a container (e.g., of a kit).

Illustrated processing block 36 provides a sensor, for example in the biocompatible carrier composition, of the carrier apparatus to detect a target in a subject and generate data corresponding to the target. The sensor may include, for example, an electrical sensor, a piezoelectric sensor, an optical sensor, and so forth. In addition, block 36 may provide a ligand functionalizing an internal portion of the carrier apparatus, an external portion of the carrier apparatus, etc., which when coupled allows the sensor to detect the target. Block 36 may involve the fabrication and/or the use of the sensor. For example, the formation of the sensor may include conventional chemical synthesis processes, molding processes, pick-and-place processes, lithography processes, printing processes, lamination processes, coating processes, deposition processes, and so forth. The utilization of the sensor may include the administration and/or the implementation of one or more sensors via one or more carrier apparatuses to provide diagnostic and/or therapeutic functionality. In addition, block 36 may involve the packaging of one or more carrier apparatuses including one or more sensors in a container (e.g., of a kit).

Illustrated processing block 38 provides a processor, for example in the biocompatible carrier composition, of the carrier apparatus that is to allow a payload to be accessible from the reservoir based on data. In addition, block 38 may allow the processor to evaluate an amount of target and allow the processor to release an amount of payload (e.g., medication payload, etc.) conditional on the amount of target. Moreover, block 38 may allow the processor to determine a context in which the carrier apparatus is to be deployed and allow the processor to release payload from a reservoir based on the context. Block 38 may involve the fabrication and/or the use of the processor. For example, the formation of the processor may involve conventional chemical synthesis processes, molding processes, pick-and-place processes, lithography processes, printing processes, lamination processes, coating processes, deposition processes, and so forth. The utilization of the processor may involve the administration and/or the implementation of one or more processors via one or more carrier apparatuses to provide diagnostic and/or therapeutic functionality. In addition, block 38 may involve the packaging of one or more carrier apparatuses including one or more processors in a container (e.g., of a kit).

Illustrated processing block 40 provides a fluidic component, for example in the biocompatible carrier composition, of the carrier apparatus to couple a sample (e.g., with the sensor) or to make payload available (e.g., from the reservoir). For example, block 40 may include providing an actuation component such as a pump, a valve, a channel (e.g., microfluidic channel, etc.), and so forth. Block 40 may involve the fabrication and/or the use of the fluidic component. For example, the formation of the fluidic component may include conventional chemical synthesis processes, molding processes, pick-and-place processes, lithography processes, printing processes, lamination processes, coating processes, deposition processes, and so forth. The utilization of the fluidic component may include the administration and/or the implementation of one or more fluidic components via one or more carrier apparatuses to provide diagnostic and/or therapeutic functionality. In addition, block 40 may involve the packaging of one or carrier apparatuses including one or more fluidic components in a container (e.g., of a kit).

Illustrated processing block 42 provides a power source, for example in the biocompatible carrier composition, of the carrier apparatus to power one or more components of the carrier apparatus. Block 42 may involve the fabrication and/or the use of the power source. For example, the formation of the power source may include conventional chemical synthesis processes, molding processes, pick-and-place processes, lithography processes, printing processes, lamination processes, coating processes, deposition processes, and so forth. The utilization of the power source may include the administration and/or the implementation of one or more power sources via one or more carrier apparatuses to provide diagnostic and/or therapeutic functionality. In addition, block 42 may involve the packaging of one or more carrier apparatuses including one or more power sources in a container (e.g., of a kit).

Illustrated processing block 44 allows payload to be accessible and/or activated. For example, medication payload, diagnostic payload, modification payload, and/or placebo payload may be accessible (e.g., to a target, to a subject, to a component of a carrier apparatus, etc.) and/or activated (e.g., transformed to a different state to provide an intended effect) by being released from the carrier apparatus, by being released into a reactor area of the carrier apparatus before being released from the carrier apparatus, by being delivered to a target, by delivering a target to the payload, by exposure to electromagnetic radiation (e.g., RF, etc.), and so forth. Block 44, therefore, may involve the administration and/or the implementation of one or more payloads via one or more carrier apparatuses to provide diagnostic and/or therapeutic functionality. In addition, block 44 may involve the packaging of one or more carrier apparatuses including one or more payloads in a container (e.g., of a kit).

While independent blocks and/or a particular order has been shown, it should be understood that one or more of the blocks may be combined and/or the method 30 may flow in any order. For example, providing the biocompatible carrier composition and the reservoir may be accomplished simultaneously and/or sequentially in any order. In another example, providing the sensor, the processor, the fluidic component, and/or the power source may be accomplished simultaneously and/or sequentially in any order. In addition, one or more blocks may be bypassed or omitted. For example, providing the sensor, the processor, the fluidic component, and/or the power source may be bypassed or omitted when the carrier apparatus responds to a passive (rather than induced) state to release payload.

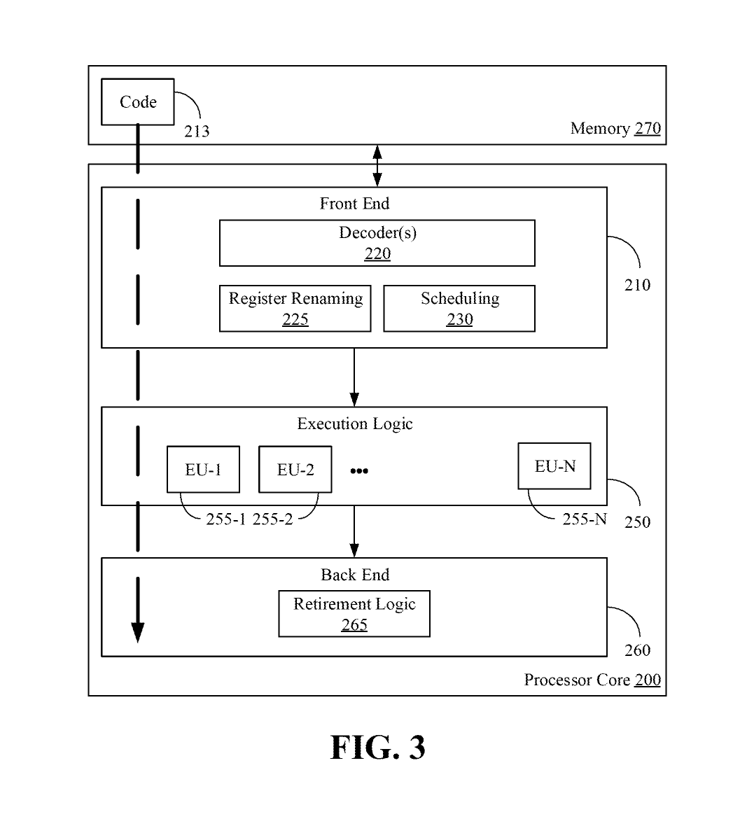

Turning now to FIG. 3, a processor core 200 is shown according to one embodiment. The processor core 200 may be the core for any type of processor, such as a micro-processor, an embedded processor, a digital signal processor (DSP), a network processor, or other device to execute code. Although only one processor core 200 is illustrated in FIG. 3, a processing element may alternatively include more than one of the processor core 200 illustrated in FIG. 3. The processor core 200 may be a single-threaded core or, for at least one embodiment, the processor core 200 may be multithreaded in that it may include more than one hardware thread context (or “logical processor”) per core.

FIG. 3 also illustrates a memory 270 coupled to the processor core 200. The memory 270 may be any of a wide variety of memories (including various layers of memory hierarchy) as are known or otherwise available to those of skill in the art. The memory 270 may include one or more code 213 instruction(s) to be executed by the processor core 200, wherein the code 213 may implement the method 30 (FIG. 2), already discussed. The processor core 200 follows a program sequence of instructions indicated by the code 213. Each instruction may enter a front end portion 210 and be processed by one or more decoders 220. The decoder 220 may generate as its output a micro operation such as a fixed width micro operation in a predefined format, or may generate other instructions, microinstructions, or control signals which reflect the original code instruction. The illustrated front end portion 210 also includes register renaming logic 225 and scheduling logic 230, which generally allocate resources and queue the operation corresponding to the convert instruction for execution.

The processor core 200 is shown including execution logic 250 having a set of execution units 255-1 through 255-N. Some embodiments may include a number of execution units dedicated to specific functions or sets of functions. Other embodiments may include only one execution unit or one execution unit that can perform a particular function. The illustrated execution logic 250 performs the operations specified by code instructions.

After completion of execution of the operations specified by the code instructions, back end logic 260 retires the instructions of the code 213. In one embodiment, the processor core 200 allows out of order execution but requires in order retirement of instructions. Retirement logic 265 may take a variety of forms as known to those of skill in the art (e.g., re-order buffers or the like). In this manner, the processor core 200 is transformed during execution of the code 213, at least in terms of the output generated by the decoder, the hardware registers and tables utilized by the register renaming logic 225, and any registers (not shown) modified by the execution logic 250.

Although not illustrated in FIG. 3, a processing element may include other elements on chip with the processor core 200. For example, a processing element may include memory control logic along with the processor core 200. The processing element may include I/O control logic and/or may include I/O control logic integrated with memory control logic. The processing element may also include one or more caches.

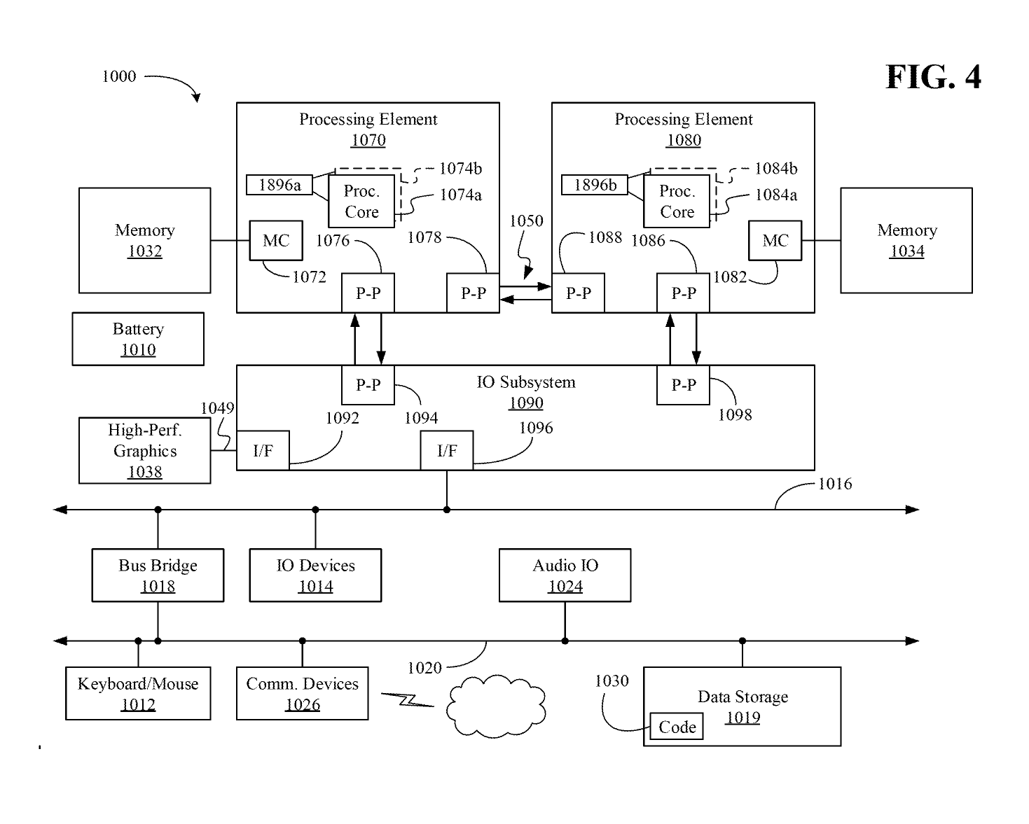

Referring now to FIG. 4, shown is a block diagram of a computing system 1000 embodiment in accordance with an embodiment. Shown in FIG. 4 is a multiprocessor system 1000 that includes a first processing element 1070 and a second processing element 1080. While two processing elements 1070 and 1080 are shown, it is to be understood that an embodiment of the system 1000 may also include only one such processing element.

The system 1000 is illustrated as a point-to-point interconnect system, wherein the first processing element 1070 and the second processing element 1080 are coupled via a point-to-point interconnect 1050. It should be understood that any or all of the interconnects illustrated in FIG. 4 may be implemented as a multi-drop bus rather than point-to-point interconnect.

As shown in FIG. 4, each of processing elements 1070 and 1080 may be multicore processors, including first and second processor cores (i.e., processor cores 1074a and 1074b and processor cores 1084a and 1084b). Such cores 1074a, 1074b, 1084a, 1084b may be configured to execute instruction code in a manner similar to that discussed above in connection with FIG. 3.

Each processing element 1070, 1080 may include at least one shared cache 1896a, 1896b. The shared cache 1896a, 1896b may store data (e.g., instructions) that are utilized by one or more components of the processor, such as the cores 1074a, 1074b and 1084a, 1084b, respectively. For example, the shared cache 1896a, 1896b may locally cache data stored in a memory 1032, 1034 for faster access by components of the processor. In one or more embodiments, the shared cache 1896a, 1896b may include one or more mid-level caches, such as level 2 (L2), level 3 (L3), level 4 (L4), or other levels of cache, a last level cache (LLC), and/or combinations thereof.

While shown with only two processing elements 1070, 1080, it is to be understood that the scope of the embodiments are not so limited. In other embodiments, one or more additional processing elements may be present in a given processor. Alternatively, one or more of processing elements 1070, 1080 may be an element other than a processor, such as an accelerator or a field programmable gate array. For example, additional processing element(s) may include additional processors(s) that are the same as a first processor 1070, additional processor(s) that are heterogeneous or asymmetric to processor a first processor 1070, accelerators (such as, e.g., graphics accelerators or digital signal processing (DSP) units), field programmable gate arrays, or any other processing element. There can be a variety of differences between the processing elements 1070, 1080 in terms of a spectrum of metrics of merit including architectural, micro architectural, thermal, power consumption characteristics, and the like. These differences may effectively manifest themselves as asymmetry and heterogeneity amongst the processing elements 1070, 1080. For at least one embodiment, the various processing elements 1070, 1080 may reside in the same die package.

The first processing element 1070 may further include memory controller logic (MC) 1072 and point-to-point (P-P) interfaces 1076 and 1078. Similarly, the second processing element 1080 may include a MC 1082 and P-P interfaces 1086 and 1088. As shown in FIG. 5, MC's 1072 and 1082 couple the processors to respective memories, namely a memory 1032 and a memory 1034, which may be portions of main memory locally attached to the respective processors. While the MC 1072 and 1082 is illustrated as integrated into the processing elements 1070, 1080, for alternative embodiments the MC logic may be discrete logic outside the processing elements 1070, 1080 rather than integrated therein.

The first processing element 1070 and the second processing element 1080 may be coupled to an I/O subsystem 1090 via P-P interconnects 1076 1086, respectively. As shown in FIG. 5, the I/O subsystem 1090 includes P-P interfaces 1094 and 1098. Furthermore, I/O subsystem 1090 includes an interface 1092 to couple I/O subsystem 1090 with a high performance graphics engine 1038. In one embodiment, bus 1049 may be used to couple the graphics engine 1038 to the I/O subsystem 1090. Alternately, a point-to-point interconnect may couple these components.

In turn, I/O subsystem 1090 may be coupled to a first bus 1016 via an interface 1096. In one embodiment, the first bus 1016 may be a Peripheral Component Interconnect (PCI) bus, or a bus such as a PCI Express bus or another third generation I/O interconnect bus, although the scope of the embodiments are not so limited.

As shown in FIG. 4, various I/O devices 1014 (e.g., cameras, sensors, etc.) may be coupled to the first bus 1016, along with a bus bridge 1018 which may couple the first bus 1016 to a second bus 1020. In one embodiment, the second bus 1020 may be a low pin count (LPC) bus. Various devices may be coupled to the second bus 1020 including, for example, a keyboard/mouse 1012, communication device(s) 1026 (which may in turn be in communication with a computer network), and a data storage unit 1019 such as a disk drive or other mass storage device which may include code 1030, in one embodiment. The illustrated code 1030 may implement the method 30 (FIG. 2), already discussed, and may be similar to the code 213 (FIG. 3), already discussed. Further, an audio I/O 1024 may be coupled to second bus 1020 and a battery 1010 may supply power to the computing system 1000.

Note that other embodiments are contemplated. For example, instead of the point-to-point architecture of FIG. 4, a system may implement a multi-drop bus or another such communication topology. Also, the elements of FIG. 4 may alternatively be partitioned using more or fewer integrated chips than shown in FIG. 4.

### Additional Notes and Examples

Example 1 may include a carrier apparatus to deliver a substance, comprising a reservoir to hold a payload, a sensor to detect a target in a subject and generate data corresponding to the target, a processor to allow the payload to be accessible from the reservoir based on the data, and a biocompatible carrier composition to hold the reservoir, the sensor, and the processor.

Example 2 may include the carrier apparatus of Example 1, further including a ligand functionalizing one or more of an internal portion of the carrier apparatus or an external portion of the carrier apparatus which when coupled is to allow the sensor to detect the target.

Example 3 may include the carrier apparatus of any one of Examples 1 to 2, further including a pump to one or more of couple a sample with the sensor or make available the payload from the reservoir.

Example 4 may include the carrier apparatus of any one of Examples 1 to 3, further including a wireless rechargeable battery to power one or more components of the carrier apparatus.

Example 5 may include the carrier apparatus of any one of Examples 1 to 4, wherein the processor is to evaluate an amount of target, and allow an amount at least of medication payload to be released conditional on the amount of the target.

Example 6 may include the carrier apparatus of any one of Examples 1 to 5, wherein the processor is to determine a context in which the carrier apparatus is to be deployed, and allow the payload to be released from the reservoir based on the context.

Example 7 may include the carrier apparatus of any one of Examples 1 to 6, wherein the reservoir is to include one or more of a placebo reservoir to hold a placebo payload, a medication reservoir to hold a medication payload to address a condition associated with the target, a diagnostic reservoir to hold a diagnostic payload to augment detection of the target, or a modification reservoir to hold a modification payload to modify at least a portion of the carrier apparatus to make available one or more of the medication payload or the diagnosis payload.

Example 8 may include the carrier apparatus of any one of Examples 1 to 7, wherein the medication reservoir is to include a first medication reservoir to hold a first medication payload to be released from the first medication reservoir to address a first condition associated with the target, and a second medication reservoir to hold a second medication payload to be released from the second medication reservoir to address one or more of the first condition associated with the target, a second condition associated with the target, or a third condition associated with another target.

Example 9 may include the carrier apparatus of any one of Examples 1 to 8, wherein the diagnostic reservoir is to include one or more of an amplifier diagnostic reservoir to hold an amplifier payload to couple with the target to amplify a characteristic of the target to be detected by the sensor via the characteristic, a reporter diagnostic reservoir to hold a reporter payload to couple with the target to be detected by the sensor via the reporter, or a synthesis diagnostic reservoir to hold a synthesis payload to couple with the target to synthesize the reporter.

Example 10 may include the carrier apparatus of any one of Examples 1 to 9, wherein the modification reservoir is to include one or more of a coating modification reservoir to hold a coating modification payload to be released from the coating modification reservoir to modify a coating of the carrier apparatus, or a matrix modification reservoir to hold a matrix modification payload to be released from the matrix modification reservoir to modify a matrix of the carrier apparatus.

Example 11 may include least one computer readable storage medium comprising a set of instructions, which when executed by a device, cause the device to provide a sensor in a biocompatible carrier composition of a carrier apparatus to detect a target in a subject and generate data corresponding to the target, and provide a processor in the biocompatible carrier composition of the carrier apparatus to allow a payload to be accessible from a reservoir in the biocompatible carrier composition of the carrier apparatus based on the data.

Example 12 may include the at least one computer readable storage medium of Example 11, wherein the instructions, when executed, cause the device to provide a ligand functionalizing one or more of an internal portion of the carrier apparatus or an external portion of the carrier apparatus which when coupled is to allow the sensor to detect the target.

Example 13 may include the at least one computer readable storage medium of any one of Examples 11 to 12, wherein the instructions, when executed, cause the device to provide a pump to one or more of couple a sample with the sensor or make available the payload from the reservoir.

Example 14 may include the at least one computer readable storage medium of any one of Examples 11 to 13, wherein the instructions, when executed, cause the device to provide a wireless rechargeable battery to power one or more components of the carrier apparatus.

Example 15 may include the at least one computer readable storage medium of any one of Examples 11 to 14, wherein the instructions, when executed, cause the device to allow the processor to evaluate an amount of target, and allow the processor to release an amount at least of medication payload conditional on the amount of the target.

Example 16 may include the at least one computer readable storage medium of any one of Examples 11 to 15, wherein the instructions, when executed, cause the device to allow the processor to determine a context in which the carrier apparatus is to be deployed, and allow the processor to release the payload from the reservoir based on the context.

Example 17 may include the at least one computer readable storage medium of any one of Examples 11 to 16, wherein the instructions, when executed, cause the device to one or more of form a placebo reservoir to hold a placebo payload, release the placebo payload from the placebo reservoir, form a medication reservoir to hold a medication payload to address a condition associated with the target, release the medication payload from the medication reservoir of the carrier apparatus to address the condition associated with the target, form a diagnostic reservoir to hold a diagnostic payload to augment detection of the target, release the diagnostic payload from the diagnostic reservoir of the carrier apparatus to augment detection of the target, form a modification reservoir to hold a modification payload to be released from the modification reservoir to modify at least a portion of the carrier apparatus to make available one or more of the medication payload or the diagnosis payload, or release the modification payload from the modification reservoir of the carrier apparatus to modify at least the portion of the carrier apparatus to make available one or more of the medication payload or the diagnosis payload.

Example 18 may include the at least one computer readable storage medium of any one of Examples 11 to 17, wherein the instructions, when executed, cause the device to one or more of form a first medication reservoir to hold a first medication payload to be released from a first medication reservoir to address a first condition associated with the target, or release the first medication payload from the first medication reservoir of the carrier apparatus to address the first condition associated with the target.

Example 19 may include the at least one computer readable storage medium of any one of Examples 11 to 18, wherein the instructions, when executed, cause the device to one or more of form a second medication reservoir to hold a second medication payload to be released from the second medication reservoir to address one or more of the first condition associated with the target, a second condition associated with the target, or a third condition associated with another target, or release the second medication payload from the second medication reservoir of the carrier apparatus to address one or more of the first condition associated with the target, the second condition associated with the target, or the third condition associated with another target.

Example 20 may include the at least one computer readable storage medium of any one of Examples 11 to 19, wherein the instructions, when executed, cause the device to one or more of form an amplifier diagnostic reservoir to hold an amplifier payload to couple with the target to amplify a characteristic of the target to be detected by the sensor via the characteristic, or make available the amplifier payload from the amplifier diagnostic reservoir of the carrier apparatus to couple with the target to amplify the characteristic of the target to be detected by the sensor via the characteristic.

Example 21 may include the at least one computer readable storage medium of any one of Examples 11 to 20, wherein the instructions, when executed, cause the device to one or more of, form a reporter diagnostic reservoir to hold a reporter payload to couple with the target to be detected by the sensor via the reporter, or make available the reporter payload from the reporter diagnostic reservoir of the carrier apparatus to couple with the target to be detected by the sensor via the reporter.

Example 22 may include the at least one computer readable storage medium of any one of Examples 11 to 21, wherein the instructions, when executed, cause the device to one or more of form a synthesis diagnostic reservoir to hold a synthesis payload to couple with the target to synthesize the reporter, or make available the synthesis payload from the synthesis diagnostic reservoir of the carrier apparatus to couple with the target to synthesize the reporter.

Example 23 may include the at least one computer readable storage medium of any one of Examples 11 to 22, wherein the instructions, when executed, cause the device to one or more of form one or more of a coating modification reservoir to hold a coating modification payload to be released from the coating modification reservoir to modify a coating of the carrier apparatus or a matrix modification reservoir to hold a matrix modification payload to be released from the matrix modification reservoir to modify a matrix of the carrier apparatus, or release one or more of the coating modification payload from the coating modification reservoir of the carrier apparatus to modify the coating of the carrier apparatus or the matrix modification payload from the matrix modification reservoir of the carrier apparatus to modify the matrix of the carrier apparatus.

Example 24 may include a method to deliver a substance, comprising providing a sensor in a biocompatible carrier composition of a carrier apparatus to detect a target in a subject and generate data corresponding to the target, and providing a processor in the biocompatible carrier composition of the carrier apparatus to allow a payload to be accessible from a reservoir in the biocompatible carrier composition of the carrier apparatus based on the data.

Example 25 may include the method of Example 24, further including providing a ligand functionalizing one or more of an internal portion of the carrier apparatus or an external portion of the carrier apparatus which when coupled is to allow the sensor to detect the target.

Example 26 may include the method of any one of Examples 24 to 25, further including providing a pump to one or more of couple a sample with the sensor or making available the payload from the reservoir.

Example 27 may include the method of any one of Examples 24 to 26, further including providing a wireless rechargeable battery to power one or more components of the carrier apparatus.

Example 28 may include the method of any one of Examples 24 to 27, further including allowing the processor to evaluate an amount of target, and allowing the processor to release an amount at least of medication payload conditional on the amount of the target.

Example 29 may include the method of any one of Examples 24 to 28, further including allowing the processor to determine a context in which the carrier apparatus is to be deployed, and allowing the processor to release the payload from the reservoir based on the context.

Example 30 may include the method of any one of Examples 24 to 29, further including one or more of forming a placebo reservoir to hold a placebo payload, releasing the placebo payload from the placebo reservoir, forming a medication reservoir to hold a medication payload to address a condition associated with the target, releasing the medication payload from the medication reservoir of the carrier apparatus to address the condition associated with the target, forming a diagnostic reservoir to hold a diagnostic payload to augment detection of the target, releasing the diagnostic payload from the diagnostic reservoir of the carrier apparatus to augment detection of the target, forming a modification reservoir to hold a modification payload to be released from the modification reservoir to modify at least a portion of the carrier apparatus to make available one or more of the medication payload or the diagnosis payload, or releasing the modification payload from the modification reservoir of the carrier apparatus to modify at least the portion of the carrier apparatus to make available one or more of the medication payload or the diagnosis payload.

Example 31 may include the method of any one of Examples 24 to 30, further including one or more of forming a second medication reservoir to hold a second medication payload to be released from the second medication reservoir to address one or more of a first condition associated with the target, a second condition associated with the target, or a third condition associated with another target, or releasing the second medication payload from the second medication reservoir of the carrier apparatus to address one or more of the first condition associated with the target, the second condition associated with the target, or the third condition associated with another target.

Example 32 may include the method of any one of Examples 24 to 31, further including one or more of forming an amplifier diagnostic reservoir to hold an amplifier payload to couple with the target to amplify a characteristic of the target to be detected by the sensor via the characteristic, or making available the amplifier payload from the amplifier diagnostic reservoir of the carrier apparatus to couple with the target to amplify the characteristic of the target to be detected by the sensor via the characteristic.

Example 33 may include the method of any one of Examples 24 to 32, further including one or more of forming a reporter diagnostic reservoir to hold a reporter payload to couple with the target to be detected by the sensor via the reporter, or making available the reporter payload from the reporter diagnostic reservoir of the carrier apparatus to couple with the target to be detected by the sensor via the reporter.

Example 34 may include the method of any one of Examples 24 to 33, further including one or more of forming a reporter diagnostic reservoir to hold a reporter payload to be released from the reporter diagnostic reservoir to couple with the target to be detected by the sensor via the reporter, or releasing the reporter payload from the reporter diagnostic reservoir of the carrier apparatus to couple with the target to be detected by the sensor via the reporter.

Example 35 may include the method of any one of Examples 24 to 34, further including one or more of forming a synthesis diagnostic reservoir to hold a synthesis payload to couple with the target to synthesize the reporter, or making available the synthesis payload from the synthesis diagnostic reservoir to couple with the target to synthesize the reporter.

Example 36 may include the method of any one of Examples 24 to 35, further including one or more of forming one or more of a coating modification reservoir to hold a coating modification payload to be released from the coating modification reservoir to modify a coating of the carrier apparatus or a matrix modification reservoir to hold a matrix modification payload to be released from the matrix modification reservoir to modify a matrix of the carrier apparatus, or releasing one or more of the coating modification payload from the coating modification reservoir of the carrier apparatus to modify the coating of the carrier apparatus or the matrix modification payload from the matrix modification reservoir of the carrier apparatus to modify the matrix of the carrier apparatus.

Example 37 may include a carrier apparatus to provide a substance comprising means for performing the method of any one of Examples 24 to 36.

Example 38 may include a kit, comprising a container and a carrier apparatus disposed in the container including a reservoir to hold a payload, a sensor to detect a target in a subject and generate data corresponding to the target, a processor to allow the payload to be accessible from the reservoir based on the data, and a biocompatible carrier composition to hold the reservoir, the sensor, and the processor.

Example 39 may include the kit of Example 38, further including an additional carrier apparatus including a diagnostic reservoir to hold a diagnostic payload to augment detection of the target, wherein the additional carrier apparatus is to be disposed in one or more of the container or another container.

Example 40 may include the kit of any one of Examples 38 to 39, further including an instruction regarding one or more of the carrier apparatus or the additional carrier apparatus.

Thus, techniques described herein may provide an ingestible capsule to perform a diagnosis of a medical condition (e.g., using a sensor and a processor) and release a drug from the same capsule into the body. In addition, the ingestible capsule may first release a substance that allows markers to be more easily detected by the same smart pill. Additional advantages may include avoiding side effects from unnecessary amounts of medication.

Techniques described herein may also provide conditional drug release from two or more reservoirs, release of an amplifier to aid in the detection of a target level (e.g., bacteria level, etc.) in the body, conditional amount of drug released based on detected substance (e.g., acid, target protein, etc.), use in clinical trials (e.g., double-blind studies), and/or wireless charging. Thus, techniques described herein may provide combined diagnostic and controllable drug release.

In particular, techniques described herein may provide a system (e.g., a capsule) to release a substance that assists in a diagnosis. For example, the capsule may release organic bacterial sensors that allow measurement of bacterial presence by producing relatively easier detectable proteins in response to the bacteria. In addition, multiple drugs may be loaded in the system to allow treatment of a differential diagnosis. Also, an amount of drug released from the system may be customized for a detected level of a chemical, bacteria, and/or other substance. The system (e.g., a smart pill) may also be used with external (to the body) location detection approaches to activate the functionality of the drug in certain areas of the body (e.g., in the digestive system). Moreover, the system may be used in a clinical trial, wherein all patients may receive a similar pill but only pills in the test group may be programmed to activate. In addition, the system may be wirelessly charged in the body via, for example, a wearable cuff.

Accordingly, techniques described herein may be utilized to detect bacteria (e.g., causing ulcers, etc.) and release an antibiotic only when the bacteria is present. In addition, stomach acid levels may be sensed and a correct level of antacid or other drug may be released. Moreover, the presence of lactic acid level (e.g., due to physical activity) may be sensed to cause a release of an anti-inflammatory drug. Also, signature substances (e.g., signature proteins, etc.) may be sensed from cancer or bacteria to illicit a correct and/or complete therapeutic response. Differential diagnosis may be provided when, for example, a subject swallows a smart pill after a meal to target too much or too little stomach acid. Also, a bacterial infection in the stomach that may lead to ulcers may be treated with antibiotics when the bacterium is present. Moreover, a bacterial infection in the intestines may be targeted, or a less than optimal digestive environment may be targeted (e.g., with the release of a bile salt, an emulsifier, etc.).

Embodiments are applicable for use with all types of semiconductor integrated circuit (“IC”) chips. Examples of these IC chips include but are not limited to processors, controllers, chipset components, programmable logic arrays (PLAs), memory chips, network chips, systems on chip (SoCs), SSD/NAND controller ASICs, and the like. In addition, in some of the drawings, signal conductor lines are represented with lines. Some may be different, to indicate more constituent signal paths, have a number label, to indicate a number of constituent signal paths, and/or have arrows at one or more ends, to indicate primary information flow direction. This, however, should not be construed in a limiting manner. Rather, such added detail may be used in connection with one or more exemplary embodiments to facilitate easier understanding of a circuit. Any represented signal lines, whether or not having additional information, may actually comprise one or more signals that may travel in multiple directions and may be implemented with any suitable type of signal scheme, e.g., digital or analog lines implemented with differential pairs, optical fiber lines, and/or single-ended lines.

Example sizes/models/values/ranges may have been given, although embodiments are not limited to the same. As manufacturing techniques (e.g., photolithography) mature over time, it is expected that devices of smaller size could be manufactured. In addition, well known power/ground connections to IC chips and other components may or may not be shown within the figures, for simplicity of illustration and discussion, and so as not to obscure certain aspects of the embodiments. Further, arrangements may be shown in block diagram form in order to avoid obscuring embodiments, and also in view of the fact that specifics with respect to implementation of such block diagram arrangements are highly dependent upon the computing system within which the embodiment is to be implemented, i.e., such specifics should be well within purview of one skilled in the art. Where specific details (e.g., circuits) are set forth in order to describe example embodiments, it should be apparent to one skilled in the art that embodiments can be practiced without, or with variation of, these specific details. The description is thus to be regarded as illustrative instead of limiting.

The term “coupled” may be used herein to refer to any type of relationship, direct or indirect, between the components in question, and may apply to electrical, mechanical, fluid, optical, electromagnetic, electromechanical or other connections. In addition, the terms “first”, “second”, etc. may be used herein only to facilitate discussion, and carry no particular temporal or chronological significance unless otherwise indicated.

As used in this application and in the claims, a list of items joined by the term “one or more of” or “at least one of” may mean any combination of the listed terms. For example, the phrases “one or more of A, B or C” may mean A; B; C; A and B; A and C; B and C; or A, B and C. In addition, a list of items joined by the term “and so forth” or “etc.” may mean any combination of the listed terms as well any combination with other terms.

Those skilled in the art will appreciate from the foregoing description that the broad techniques of the embodiments can be implemented in a variety of forms. Therefore, while the embodiments have been described in connection with particular examples thereof, the true scope of the embodiments should not be so limited since other modifications will become apparent to the skilled practitioner upon a study of the drawings, specification, and following claims.

We claim:

1. A carrier apparatus to deliver a substance comprising:
a reservoir to hold a payload;
a sensor to detect a target in a subject and generate data corresponding to the target;
a processor to allow the payload to be accessible from the reservoir based on the data; and
a biocompatible carrier composition to hold the reservoir, the sensor, and the processor.

2. The carrier apparatus of claim 1, further including a ligand functionalizing one or more of an internal portion of the carrier apparatus or an external portion of the carrier apparatus which when coupled is to allow the sensor to detect the target.

3. The carrier apparatus of claim 1, further including a pump to one or more of couple a sample with the sensor or make available the payload from the reservoir.

4. The carrier apparatus of claim 1, further including a wireless rechargeable battery to power one or more components of the carrier apparatus.

5. The carrier apparatus of claim 1, wherein the processor is to:
evaluate an amount of target; and
allow an amount at least of medication payload to be released conditional on the amount of the target.

6. The carrier apparatus of claim 1, wherein the processor is to:
determine a context in which the carrier apparatus is to be deployed; and
allow the payload to be released from the reservoir based on the context.

7. The carrier apparatus of claim 1, wherein the reservoir is to include one or more of:
a placebo reservoir to hold a placebo payload;
a medication reservoir to hold a medication payload to address a condition associated with the target;
a diagnostic reservoir to hold a diagnostic payload to augment detection of the target; or
a modification reservoir to hold a modification payload to modify at least a portion of the carrier apparatus to make available one or more of the medication payload or the diagnosis payload.

8. The carrier apparatus of claim 7, wherein the medication reservoir is to include:
a first medication reservoir to hold a first medication payload to be released from the first medication reservoir to address a first condition associated with the target; and
a second medication reservoir to hold a second medication payload to be released from the second medication reservoir to address one or more of the first condition associated with the target, a second condition associated with the target, or a third condition associated with another target.

9. The carrier apparatus of claim 7, wherein the diagnostic reservoir is to include one or more of:
an amplifier diagnostic reservoir to hold an amplifier payload to couple with the target to amplify a characteristic of the target to be detected by the sensor via the characteristic;
a reporter diagnostic reservoir to hold a reporter payload to couple with the target to be detected by the sensor via the reporter; or
a synthesis diagnostic reservoir to hold a synthesis payload to couple with the target to synthesize the reporter.

10. The carrier apparatus of claim 7, wherein the modification reservoir is to include one or more of:
a coating modification reservoir to hold a coating modification payload to be released from the coating modification reservoir to modify a coating of the carrier apparatus; or
a matrix modification reservoir to hold a matrix modification payload to be released from the matrix modification reservoir to modify a matrix of the carrier apparatus.

11. At least one computer readable storage medium comprising a set of instructions, which when executed by a device, cause the device to:
provide a sensor in a biocompatible carrier composition of a carrier apparatus to detect a target in a subject and generate data corresponding to the target; and
provide a processor in the biocompatible carrier composition of the carrier apparatus to allow a payload to be accessible from a reservoir in the biocompatible carrier composition of the carrier apparatus based on the data.

12. The at least one computer readable storage medium of claim 11, wherein the instructions, when executed, cause the device to:
allow the processor to evaluate an amount of target; and
allow the processor to release an amount at least of medication payload conditional on the amount of the target.

13. The at least one computer readable storage medium of claim 11, wherein the instructions, when executed, cause the device to:
allow the processor to determine a context in which the carrier apparatus is to be deployed; and
allow the processor to release the payload from the reservoir based on the context.

14. The at least one computer readable storage medium of claim 11, wherein the instructions, when executed, cause the device to three or more of:
form a placebo reservoir to hold a placebo payload;
release the placebo payload from the placebo reservoir;
form a medication reservoir to hold a medication payload to address a condition associated with the target;
release the medication payload from the medication reservoir of the carrier apparatus to address the condition associated with the target;
form a diagnostic reservoir to hold a diagnostic payload to augment detection of the target;
release the diagnostic payload from the diagnostic reservoir of the carrier apparatus to augment detection of the target;
form a modification reservoir to hold a modification payload to be released from the modification reservoir to modify at least a portion of the carrier apparatus to make available one or more of the medication payload or the diagnosis payload; or
release the modification payload from the modification reservoir of the carrier apparatus to modify at least the portion of the carrier apparatus to make available one or more of the medication payload or the diagnosis payload.

15. The at least one computer readable storage medium of claim 14, wherein the instructions, when executed, cause the device to one or more of:
form a first medication reservoir to hold a first medication payload to be released from a first medication reservoir to address a first condition associated with the target; or
release the first medication payload from the first medication reservoir of the carrier apparatus to address the first condition associated with the target.

16. The at least one computer readable storage medium of claim 14, wherein the instructions, when executed, cause the device to one or more of:
form a second medication reservoir to hold a second medication payload to be released from the second medication reservoir to address one or more of the first condition associated with the target, a second condition associated with the target, or a third condition associated with another target; or
release the second medication payload from the second medication reservoir of the carrier apparatus to address one or more of the first condition associated with the target, the second condition associated with the target, or the third condition associated with another target.

17. The at least one computer readable storage medium of claim 14, wherein the instructions, when executed, cause the device to one or more of:
form an amplifier diagnostic reservoir to hold an amplifier payload to couple with the target to amplify a characteristic of the target to be detected by the sensor via the characteristic; or
make available the amplifier payload from the amplifier diagnostic reservoir of the carrier apparatus to couple with the target to amplify the characteristic of the target to be detected by the sensor via the characteristic.

18. The at least one computer readable storage medium of claim 14, wherein the instructions, when executed, cause the device to one or more of:
form a reporter diagnostic reservoir to hold a reporter payload to couple with the target to be detected by the sensor via the reporter; or
make available the reporter payload from the reporter diagnostic reservoir of the carrier apparatus to couple with the target to be detected by the sensor via the reporter.

19. The at least one computer readable storage medium of claim 14, wherein the instructions, when executed, cause the device to one or more of:
form a synthesis diagnostic reservoir to hold a synthesis payload to couple with the target to synthesize the reporter; or
make available the synthesis payload from the synthesis diagnostic reservoir of the carrier apparatus to couple with the target to synthesize the reporter.

20. The at least one computer readable storage medium of claim 14, wherein the instructions, when executed, cause the device to one or more of:
form one or more of a coating modification reservoir to hold a coating modification payload to be released from the coating modification reservoir to modify a coating of the carrier apparatus or a matrix modification reservoir to hold a matrix modification payload to be released from the matrix modification reservoir to modify a matrix of the carrier apparatus; or
release one or more of the coating modification payload from the coating modification reservoir of the carrier apparatus to modify the coating of the carrier apparatus or the matrix modification payload from the matrix modification reservoir of the carrier apparatus to modify the matrix of the carrier apparatus.

21. A method to deliver a substance comprising:
providing a sensor in a biocompatible carrier composition of a carrier apparatus to detect a target in a subject and generate data corresponding to the target; and
providing a processor in the biocompatible carrier composition of the carrier apparatus to allow a payload to be accessible from a reservoir in the biocompatible carrier composition of the carrier apparatus based on the data.

22. The method of claim 21, further including:
allowing the processor to evaluate an amount of target;
allowing the processor to release an amount at least of medication payload conditional on the amount of the target;
allowing the processor to determine a context in which the carrier apparatus is to be deployed; and
allowing the processor to release the payload from the reservoir based on the context.

23. The method of claim 21, further including three or more of:
forming a placebo reservoir to hold a placebo payload;
releasing the placebo payload from the placebo reservoir;
forming a medication reservoir to hold a medication payload to address a condition associated with the target;
releasing the medication payload from the medication reservoir of the carrier apparatus to address the condition associated with the target;
forming a diagnostic reservoir to hold a diagnostic payload to augment detection of the target;
releasing the diagnostic payload from the diagnostic reservoir of the carrier apparatus to augment detection of the target;
forming a modification reservoir to hold a modification payload to be released from the modification reservoir to modify at least a portion of the carrier apparatus to make available one or more of the medication payload or the diagnosis payload; or
releasing the modification payload from the modification reservoir of the carrier apparatus to modify at least the portion of the carrier apparatus to make available one or more of the medication payload or the diagnosis payload.

24. A kit to provide a substance comprising:
a container; and
a carrier apparatus disposed in the container including:
a reservoir to hold a payload;
a sensor to detect a target in a subject and generate data corresponding to the target;
a processor to allow the payload to be accessible from the reservoir based on the data; and
a biocompatible carrier composition to hold the reservoir, the sensor, and the processor.

25. The kit of claim 24, further including an additional carrier apparatus including a diagnostic reservoir to hold a diagnostic payload to augment detection of the target, wherein the additional carrier apparatus is to be disposed in one or more of the container or another container.
